(19) United States
(12) Patent Application Publication
(10) Pub. No.: US 2007/0014687 A1
(43) Pub. Date: Jan. 18, 2007

(54) Composition and method for deactivating allergenic proteins on surfaces

(76) Inventors: Rick L. Tabor, Glenview, IL (US); James A. Hartlage, Chicago, IL (US); Daniel S. Bertholf, Glenview, IL (US); S. Ann Hartlage, Chicago, IL (US)

Correspondence Address: MCANDREWS HELD & MALLOY, LTD, 500 WEST MADISON STREET, SUITE 3400, CHICAGO, IL, 60661, US

(21) Appl. No.: 11/485,093

(22) Filed: Jul. 12, 2006

Related U.S. Application Data
(60) Provisional application No. 60/698,348, filed on Jul. 12, 2005.

(51) Int. Cl. A61L 2/18; A61K 39/35

(52) U.S. Cl. 422/37; 424/275.1

(57) ABSTRACT

The presently described technology provides for methods and compositions for deactivating the allergenic protein content of surfaces by treatment with an allergenic protein deactivating compound. Preferred allergenic protein deactivating compositions of the present technology comprise: (1) cationic compounds and (2) nitrogen-containing compounds. Quaternary ammonium salts are particularly preferred allergenic protein deactivating compounds. In some embodiments, allergenic protein deactivating formulations of the present technology additionally contain antimicrobial agents to deactivate microorganisms on surfaces, in the air, or both. Additives including cyclodextrins, soluble aluminum salts, urea, alkyl trimethyl ammonium salts, polyvinyl pyrrolidone, tannic acid, immobilized tannic acid, polyphenols, enzymes, benzyl benzoate, inorganic salts, surfactants, buffers, oils, waxes, solvents, preservatives and mixtures thereof are also envisioned for use with the described technology.

### RELATED APPLICATIONS

This application claims priority to U.S. Provisional Application Ser. No. 60/698,348, filed Jul. 12, 2005, now abandoned, the disclosure of which is hereby incorporated by reference herein in its entirety.

### FEDERALLY SPONSORED RESEARCH OR DEVELOPMENT

[Not Applicable]

### MICROFICHE/COPYRIGHT REFERENCE

[Not Applicable]

### FIELD OF THE INVENTION

The presently described technology relates generally to methods and compositions for deactivating the allergenic protein content of surfaces by treatment with an allergenic protein deactivating compound. In some embodiments, allergenic protein deactivating formulations of the present technology additionally contain antimicrobial agents to deactivate microorganisms on surfaces, in the air, or both.

### BACKGROUND OF THE INVENTION

Allergens are substances that trigger allergic reactions. Common cleaning methods are often insufficient to substantially neutralize and/or remove many of the allergens commonly present on various surfaces. As a result, various anti-allergen chemistries have been developed for use in various contexts.

Allergen neutralizers that are currently known include, for example, tannic acid and polyphenols, bleach, enzyme solutions and cyclodextrins. Tannic acid and polyphenols are inappropriate for many applications, such as for use on fabrics, because they are known to cause staining and are skin irritants. Chlorine bleach is commonly used against mold on hard surfaces such as tile, metal and plastics, but is inappropriate for use on colored fabrics because it removes coloration, and can cause skin irritation.

One commercial example of a formulation using cyclodextrin is Febreze® Allergen Reducer produced by Proctor & Gamble of Cincinnati, Ohio. The patents that are said to cover this product include one or more of U.S. Pat. Nos. 6,146,621; 5,942,217; 5,939,060; 5,783,544; 5,714,137; 5,668,097; and 5,593,670. As an example, U.S. Pat. No. 6,146,621 warns that the Proctor & Gamble formulation should not be applied to shiny surfaces including, e.g., chrome, glass, smooth vinyl, leather, shiny plastic, shiny wood, etc., because spotting and filming can more readily occur on those types of surfaces with the subject cyclodextrin-containing formulation. Additionally, the formulation is described as not being suitable for use on human skin, especially when an antimicrobial preservative is present in the composition, because skin irritation can occur.

Another example of an allergen reducing formulation is described in U.S. Patent Publication No. 20040007251, which provides for wipes used to remove an allergen from (among other things) hard surfaces, human skin and animal fur. The wipes are further described in U.S. Patent Publication No. 20040007251 as containing an additive, such as a lectin, a protease, and/or an enzyme inhibitor that can bind the allergen to the wipe such that the allergen can be removed from the surface. Patent Publication No. 20040007251 also provides that when the wipes contain a protease or an enzyme inhibitor, alone or in combination with a lectin, the protease or enzyme inhibitor may provide the additional benefit of chemically altering the allergen to reduce its allergenicity.

Another example of an allergen deactivating formulation is described in U.S. Pat. No. 6,800,247, which describes a method for deactivating Dermatophagoides farinae (Der-f) and Dermatophagoides pteronyssinus (Der-p) dust mite allergens using a variety of deactivating agents. The list of deactivants includes polyquaternium 10 (which is described as being hydroxyethyl cellulose reacted with Polymer JR-125, a trimethyl ammonium epoxide compound), polyvinyl pyrrolidone and hexadecyl trimethyl ammonium chloride. The reference further provides that some of the deactivants are specific to the type of dust mite allergen being treated. For example, an effective Der-f allergen deactivant will not automatically work effectively as a Der-p allergen deactivant. The use of polyquaterniums based on cellulose (such as polyquaternium 10) is undesirable in certain applications because these types of polyquaterniums increase the wettability of textile surfaces and subsequently reduce the stain resistance of textiles to which they are applied due to their high degree of hydrophilicity.

One commercial example of an allergen reducing enzymatic formula is a pet care product called Nature's Miracle® Dander Remover & Body Deodorizer, produced by Pets'N People, Inc., a subsidiary of Eight In One Pet Products of Hauppauge, N.Y. This product is described as a non-toxic, biodegradable enzyme formula that is designed to be sprayed onto a pet's fur or skin and wiped off with a paper towel.

Other pet care products include Allerpet® Dander Lotion and Simple Solution® Allergy Relief Wipes. Allerpet® Dander Lotion is produced by Farnam Companies, Inc., located in Phoenix, Ariz. and is described as a non-toxic, odorless and safe solution that removes the saliva, dander and urine allergens from an animal's coat and skin. Allerpet® Dander Lotion can be wiped or sprayed onto an animal and then rubbed or combed into the animal's fur. Simple Solution® Allergy Relief Wipes are produced by Bramton Company of Dallas, Tex., and are described as pre-moistened cloths that contain a blend of conditioning agents and proteins to wipe away pet dander and loose hair. However, neither of these products is described as being suitable for use on other surfaces such as textiles or hard surfaces.

In light of the above, there exists a continued need for a low toxicity, low staining, mild anti-allergenic formulation which deactivates allergenic proteins on surfaces that is preferably non-irritating to humans and animals.

### BRIEF SUMMARY OF THE INVENTION

The present invention generally relates to deactivating allergenic proteins on various surfaces by treating them with an allergenic protein deactivating agent. The present technology can be useful, for example, in the following applications: sprays for upholstery or carpet, sprays for textiles, additives for dusting sprays, a pre-treatment for newly manufactured carpet to deactivate allergenic proteins as the carpet absorbs or interacts with them, a treatment for ducts to deactivate allergenic proteins as they occur during air passage, an anti-allergenic treatment in combination with currently used biocides for mold-infested ducts, a treatment for allergen-reducing air filtration systems and home air filters, an additive for medicinal creams or lotions, a treatment for masks used by people to reduce exposure to allergens, a skin spray used by people to reduce exposure to allergens, or a treatment for allergen-producing pets.

As used herein, the terms “deactivating,” “deactivation” and “deactivate” refer to rendering allergenic proteins inactive and/or ineffective with respect to allergenic impact in humans or animals, and encompass both partial and total deactivation, as well as a reduction in or a prevention of allergenic impact. Allergen deactivation can be measured by immunoassay of the substances removed from a surface after treatment of the surface using the present technology.

At least one embodiment of the present invention provides for a method for deactivating the allergenic proteins on a surface comprising the step of exposing the surface to an allergenic protein deactivating formulation containing an allergenic protein deactivating compound. The allergenic protein deactivating compound preferably comprises poly-functional nitrogen containing compounds, mono-functional nitrogen-containing compounds, poly-functional cation-containing compounds, mono-functional cation-containing compounds, combinations thereof, or derivatives thereof. Methods of exposing surfaces to an allergenic protein deactivating formulation of the present technology include, for example, spraying or wiping the surface with a dilute solution of the allergenic protein deactivating compound.

Solutions of the present technology are intended to be safe for contact with humans and animals, and provide deactivation of allergenic proteins on the surfaces to which they are applied. The solutions preferably contain a reduced level of particulate material as compared to various other conventional anti-allergenic formulations. More preferably, the solutions contain a low level of particulate material (i.e. the allergenic deactivating agent). Specifically, it is preferred that these solutions contain no more than about 5% by weight of particulate materials. More preferably, solutions contain about 1% or less of the particulate matter.

Accordingly, in at least one embodiment of the present technology, a method for deactivating the allergenic proteins on a surface is provided, wherein the method comprises the step of exposing the surface to an allergenic protein deactivating formulation further comprising an allergenic protein deactivating compound, wherein the allergenic protein deactivating compound comprises a concentration of about 5% by weight or less of a particulate material in a solvent, a diluent, or an aqueous delivery composition.

Surfaces on which the present technology may be used include textiles, leather, fur, non-wovens, and wood and other hard surfaces. In order to avoid streaking and filming on hard or smooth surfaces, it is desired that the concentration of the allergenic deactivating agent or blend of agents be less than about 5% by weight in the solvent, aqueous composition, or diluent used to deliver the agent or agents. Preferably, the concentration of the allergenic deactivating agent or blend of agents is less than about 2.5% by weight, less than about 2% by weight, or less than about 1.5% by weight. Most preferably, the concentration of the allergenic deactivating agent or blend of agents is about 1% by weight, or less than about 1% by weight. Suitable amounts of an allergenic deactivating agent or blend of agents that are less than about 1% include, for example, concentrations up to about 0.5% by weight, up to about 0.2% by weight, up to about 0.05% by weight or up to about 0.025% by weight.

The methods and compositions of the present technology can further comprise antimicrobial agents for deactivating microorganisms on surfaces, in the air, or both.

Diluents suitable for use with the present technology include, for example, water, alcohols, glycols and other organic solvents. Water is a preferred diluent. Diluents can be utilized alone or in combination with one another without departing from the spirit and scope of the present described technology.

A further embodiment of the presently described technology provides for an allergen deactivating formulation with a low toxicity level, suitable for use in contact with human skin or animal fur and/or skin. In at least one aspect of this embodiment, an allergen deactivating formulation is provided that will not cause staining on textiles or other surfaces. Further, allergen deactivating formulations of this embodiment preferably have a low, reduced or zero level of particulates, as described above.

In one embodiment, an allergenic protein deactivating formulation is provided that comprises an allergenic protein deactivating compound selected from the group consisting essentially of sulfobetaines, betaines, amino carboxylates, amine oxides, amides, nitrogen-containing heterocyclic amides, dimethyl dialkyl ammonium salts, methyl trialkyl ammonium salts, tetraalkyl ammonium salts, tetramethyl ammonium salts, imidazoline-based quaternaries, benzyl alkyl dimethyl ammonium salts, benzyl methyl dialkyl ammonium salts, benzyl trialkyl ammonium salts, dibenzyl dimethyl ammonium salts, propoxylated quaternary amines, ethoxylated quaternary amines, dialkyl diethoxylated ammonium salts, dipropoxylated ammonium salts, alkyl methyl diethoxylated ammonium salts, dipropoxylated ammonium salts, tetraethoxylated ammonium salts, tetrapropoxylated ammonium salts, methyl triethoxylated ammonium salts, tripropoxylated ammonium salts, ester quaternaries, phosphate-containing quaternaries, pyridinium salts, polyvinyl pyridinium salts, vinyl pyridinium copolymer salts, nitrogen-containing heterocyclic amine salts, non-cellulose-based polyquaternium salts, amino carboxylates, amine oxides, amides, nitrogen-containing heterocyclic amides, vinyl pyrrolidone copolymers, derivatives thereof, and combinations thereof.

### BRIEF DESCRIPTION OF SEVERAL VIEWS OF THE DRAWINGS

[Not Applicable]

### DETAILED DESCRIPTION OF THE INVENTION

The presently described technology provides methods and compositions for deactivating the allergenic protein content of surfaces by treatment with an allergenic protein deactivating compound. For example, allergenic protein deactivating compositions of the present technology preferably comprise: (1) cationic compounds and (2) nitrogen-containing compounds. As another example, the technology of the present invention includes exposing a surface to an allergenic protein deactivating formulation. This exposure deactivates allergenic protein content present on the surface to which it is applied. Surfaces on which the present technology may be used include, for example, textiles, leather, fur, non-wovens, and wood and other hard surfaces.

Allergenic protein deactivating compounds used in formulations of the present technology can comprise, for example, poly-functional or mono-functional nitrogen-containing or cation-containing compounds, as well as combinations or derivatives thereof. Examples of allergenic protein deactivating compounds include, for example, quaternium salts, polyquaternium salts, sulfobetaines, betaines, amino carboxylates, amine oxides, amides, nitrogen-containing heterocyclic amides, and mixtures thereof. Quaternium salts can include, for example, the following: dimethyl dialkyl ammonium salts, methyl trialkyl ammonium salts, tetraalkyl ammonium salts, tetramethyl ammonium salts, imidazoline based quaternaries, benzyl alkyl dimethyl ammonium salts, benzyl methyl dialkyl ammonium salts, benzyl trialkyl ammonium salts, dibenzyl dimethyl ammonium salts, propoxylated or ethoxylated quaternary amines, dialkyl diethoxylated or dipropoxylated ammonium salts, alkyl methyl diethoxylated or dipropoxylated ammonium salts, tetraethoxylated or tetrapropoxylated ammonium salts, methyl triethoxylated or tripropoxylated ammonium salts, ester quaternaries, phosphate-type quaternaries, pyridinium salts, polyvinyl pyridinium salts, vinyl pyridinium copolymer salts, and nitrogen-containing heterocyclic amine salts, and non-cellulose-based polyquaternium salts, vinyl pyrrolidone copolymers, derivatives thereof, and combinations thereof.

Particularly preferred allergenic protein deactivating compounds comprise quaternary ammonium salts, such as, for example, non-cellulose-based polymeric quaternary amine salts, dimethyl dialkyl ammonium chloride, and dimethyl ditallow ammonium chloride. For example, in one embodiment, an allergenic protein deactivating formulation comprises an allergenic protein deactivating compound comprising at least about 0.2%, or at least about 0.05%, by weight dimethyl ditallow ammonium chloride. In another embodiment, an allergenic protein deactivating formulation comprises an allergenic protein deactivating compound comprising at least about 0.2%, or at least about 0.05%, by weight of a non-cellulose-based polymeric quaternary amine salt. In another embodiment, an allergenic protein deactivating formulation comprises an allergenic protein deactivating compound comprising 0.2%, or at least about 0.05%, by weight dimethyl didecyl ammonium chloride.

In some embodiments, allergenic protein deactivating compounds comprise mixtures of components. For example, in one embodiment, an allergenic protein deactivating compound comprises at least about 0.2% by weight, or at least about 0.05% by weight, of a quaternary amine salt mixture comprising: at least about 32% n-alkyl (50% C14, 40% C12, 10% C16) dimethyl benzyl ammonium chloride, at least about 24% n-octyl decyl dimethyl ammonium chloride, at least about 12% di-n-octyl dimethyl ammonium chloride, and at least about 12% di-n-decyl dimethyl ammonium chloride.

It is known that deposition of additives can cause streaking and filming on hard, smooth surfaces and can also cause an increased tendency towards soiling on textile surfaces. Hence, levels of allergenic protein deactivating agents which avoid or substantially reduce these undesirable side effects, while still remaining efficacious at deactivating the allergenic proteins on the surface in question, are preferred. Formulations of the presently described technology thus preferably utilize as low a level of the above agents in the final allergenic protein deactivation formulation as will be effective, in order to avoid excessive deposition of the allergenic protein deactivating compound onto the surface being treated. It is desired that the concentration of the allergenic deactivating agent, or blend of agents, be about 5% by weight, or less than about 5% by weight in the solvent, aqueous composition, or diluent used to deliver the agent or agents. Preferably, the concentration of the allergenic deactivating agent or blend of agents is less than about 2.5% by weight, less than about 2% by weight, or less than about 1.5% by weight. Most preferably, the concentration of the allergenic deactivating agent or blend of agents is about 1% by weight, or less than about 1% by weight. Suitable amounts of an allergenic deactivating agent or blend of agents that are less than about 1% include, for example, concentrations up to about 0.5% by weight, up to about 0.2% by weight, up to about 0.05% by weight or up to about 0.025% by weight.

The most preferred allergenic protein deactivating compounds of the presently described technology are quaternary ammonium salts and combinations thereof. Although not wanting to be bound by any particular theory, it is believed that quaternary ammonium salts of the presently described technology utilized in at least one method of the present invention may operate in a similar manner and that they may denature allergenic proteins on textiles and surfaces by chemically associating with the allergenic proteins to change their surface chemistry from protein-like to hydrocarbon-like.

Further, it is believed that the complexed allergenic proteins may remain on the textile or surface, but do not readily activate antibodies in humans because their molecular shapes have been altered in a process known as denaturation (see, A. L. Lehninger, Biochemistry, 1975, Worth Publishers, page 62, for a description of protein denaturation). It is known that antibodies or immunoglobulins form in the blood serum in response to the introduction of a foreign protein. In the case of allergenic proteins, the immunoglobulin formed is known as IgE, or Immunoglobulin E. People with allergies produce an excess of IgE, which collects around certain types of cells in the body. When people with allergies are exposed to allergenic proteins, IgE forms an antigen-antibody complex, which causes the release of chemicals such as histamine from the nearby cells. These chemicals, in turn, can cause runny noses, bronchial spasms, asthma, itching, hives, nausea and even anaphylactic shock and death. Hence, it is also believed that if the surface or shape of an allergenic protein can be changed by complexation with the allergenic protein deactivating compounds of the presently described technology, then the ability of the allergenic protein to activate antibodies is reduced or negated.

Other described allergenic protein deactivating compounds of the present technology (i.e., other than the quaternary ammonium salts) are also believed to provide surface chemistry changes in the allergenic proteins from protein-like to hydrocarbon-like, as described above.

In some embodiments, protein deactivating formulations of the present technology further comprise at least one antimicrobial agent. The term antimicrobial agent as used herein refers to an agent that deactivates microorganisms, and renders them inactive and/or ineffective with respect to their harmful impact in humans or animals. The term antimicrobial agent encompasses both partial and total deactivation, as well as a reduction in or a prevention of harmful impact by the microorganisms. The term antimicrobial agent also encompasses partial or total destruction of microorganisms as well as partial or total inhibition of the growth of microorganisms. Examples of types of antimicrobial agents include, but are not limited to disinfectants, sanitizers and antiseptics. The types of microorganisms deactivated by antimicrobial compounds can be spores, vegetative microorganisms, or a combination of both.

In some embodiments, antimicrobial agents suitable for use with the present technology are air treating antimicrobial agents. Air treating antimicrobial agents can be used, for example, in spray applications, to deactivate microorganisms in the air. In such applications, allergenic deactivating formulations of the present technology containing air treating antimicrobial agents can be dispensed, for example, by aerosolizing droplet particles through an actuator valve. Glycols are a particularly preferred category of air treating antimicrobial agents. Some examples of glycols suitable for use with the present technology include, but are not limited to, propylene glycol, dipropylene glycol, triethylene glycol, derivatives thereof, and mixtures thereof.

In other embodiments, antimicrobial agents suitable for use with the present technology are surface treating antimicrobial agents. Surface treating antimicrobial agents suitable for use with the present technology include antimicrobial agents for use on textiles, leather, fur, non-wovens, wood and other hard surfaces, and soft or porous surfaces. Some examples of surface treating antimicrobial agents suitable for use with the present technology include, but are not limited to, synthetic detergents such as quaternary ammonium compounds and sodium alkylbenzene sulfonates, mercury compounds, halogens, halogen compounds, aldehydes, phenolics, isothiazonlines, alcohols, carbamates, halide compounds, peroxides, parabens, iodine, metals, peracids, carbonates, hypochlorites, chloramines, benzalkonium chloride, derivatives thereof, and mixtures thereof. In some preferred embodiments, the surface treating antimicrobial agents are quaternary ammonium salts such as quaternary ammonium chlorides and quaternary ammonium saccharinates, metal salts such as silver salts, or alcohols such as isopropanol.

In certain embodiments, allergenic protein deactivating formulations of the present technology comprise at least one allergenic protein deactivating compound. In other embodiments, allergenic protein deactivating formulations of the present technology comprise at least one allergenic protein deactivating compound and at least one antimicrobial agent. In some preferred embodiments, allergenic protein deactivating formulations comprise at least one allergenic protein deactivating compound and at least one air treating antimicrobial agent. In other preferred embodiments, allergenic protein deactivating formulations comprise at least one allergenic protein deactivating compound and at least one surface treating antimicrobial agent. In still other preferred embodiments, allergenic protein deactivating formulations comprise at least one allergenic protein deactivating compound, at least one surface treating antimicrobial agent, and at least one air treating antimicrobial agent.

Diluents suitable for use with the present technology include, for example, water, alcohols, glycols and other organic solvents. Water is a particularly preferred diluent. Diluents can be utilized alone or in combination with one another without departing from the spirit and scope of the present described technology.

It is anticipated that the efficacy of the allergenic protein deactivating formulation(s) of the presently described technology may be improved when further combined with additives such as, for example, known allergenic protein deactivating substances, surfactants, buffers, oils and waxes, solvents, preservatives or mixtures thereof.

Examples of surfactant classes suitable for use with the present technology include, for example, nonionic surfactants, cationic surfactants, amphoteric surfactants, anionic surfactants, and combinations thereof. Although many of the compounds of the presently described technology are surfactants, the use of a secondary surfactant along with the allergen deactivating agents of the present technology is anticipated to provide improved wetting and penetration of surfaces onto which allergens might be absorbed. Improved wetting and penetration of surfaces and textiles will allow for improved delivery of the allergenic deactivating agent to the allergen.

Examples of nonionic surfactants include, for example, alcohol ethoxylates, alkylphenol ethoxylated, ethylene oxide/propylene oxide block copolymers, alkyl polyglycosides, alkanol amides, amine ethoxylates, quaternary ammonium compounds, amine oxides, polyamines, alkoxylated silicones, and combinations thereof.

Examples of anionic surfactants include, but are not limited to, linear alkylbenzene sulfonates, alcohol sulfates, alcohol ether sulfates, metal salts of long chain fatty acids, secondary alkane sulfonates, alpha olefin sulfonates, methyl ester sulfonates, phosphate esters, dialkyl sulfosuccinates, alkyl diphenyl oxide disulfonates, naphthalene sulfonates, lignosulfonates, and combinations thereof.

Examples of cationic surfactants include, for example, quaternary alkyl amines, polyquaterniums, quaternary aromatic or heterocyclic amines, and combinations thereof.

Examples of amphoteric surfactants include, for example betaines, amine oxides, sultaines, and combinations thereof.

Known allergenic protein deactivating substances with which the current technology may be combined to produce additive or synergistic allergenic protein deactivation against multiple allergenic protein types include, but are not limited to: cyclodextrins, soluble aluminum salts, urea, alkyl trimethyl ammonium salts, polyvinyl pyrrolidone, tannic acid, immobilized tannic acid, polyphenols, enzymes, benzyl benzoate, inorganic salts, derivatives thereof, and combinations thereof.

One use of buffers in the present technology is to maintain a pH in a desirable range in formulations for the purpose of stability, solubility or efficacy. Buffers are defined as a solution containing both a weak acid and its conjugate weak base. There are hundreds of existing buffer systems. A few examples of buffer systems include, for example, triethylammonium bicarbonate, triethylammonium formate, acetate/acetic acid, carbonate/bicarbonate, potassium phosphate/phosphoric acid, and ammonium chloride/ammonium hydroxide.

Oils and waxes may be used in furniture polish compositions of the present technology to aid in dust removal from surfaces and to help maintain a glossy appearance on hard surfaces. Examples of waxes include without limitation hydrocarbon materials which have crystallinity at room temperature, natural and synthetic waxes, ester-type waxes, microcrystalline waxes, petroleum waxes, polyethylenes, polypropylenes, candililla wax, and carnuba wax. Examples of oils include, for example, mineral oils, vegetable oils, linseed oil, tung oil, soybean oil, olive oil, sunflower seed oil, castor oil, canola oil, corn oil, cottonseed oil, lemon oil, orange oil, silicone oil, polydialkyl siloxanes, oligomers and polymers of alpha olefins, paraffin oil, and esters of fatty acids.

Solvents may be used with the present technology as diluents, solubilizing agents, and as a means to provide a convenient liquid delivery. Solvents used for the practice of the presently described technology are preferably liquid under ambient conditions. Examples of suitable solvents include, for example, water, methanol, ethanol, isopropanol, alcohols, ethers, esters, amides, alkanes, halogenated hydrocarbons, silicones, oils and the like.

Preservatives may be used with the present technology to provide biocidal activity in formulations of the present technology to prevent growth of mold, bacteria, fungus, and other undesirable biological substances during storage, prevent oxidation or degradation of the product and extend the useful life of the final product. Examples of preservatives include, but are not limited to, formaldehyde, antioxidants, biocides, aldhehydes, fungicides, chlorinated hydrocarbons, organometallics, metal salts, organic sulfur compounds, phenolics, quaternary ammonium compounds, and combinations thereof.

All documents, e.g., patents and journal articles, cited above and/or below, are hereby incorporated by reference in their entirety.

Certain embodiments of the presently described technology are illustrated in the following examples, which are not to be construed as limiting the invention or scope of the methods or compositions described herein. One skilled in the art will recognize that modifications may be made in the presently described technology without deviating from the spirit or scope of the invention. All levels and ranges, temperatures, results, etc., used and/or described herein are approximations unless otherwise specified. Additionally, all percentages are weight percentages unless otherwise specified.

The following Examples demonstrate some of the advantages of deactivating allergenic protein content using several types of allergenic protein deactivating compounds.

### EXAMPLE 1

The allergens used in this experiment were obtained from dust mite and cockroach sources. Purified standard was used for the dust mite allergen. The cockroach allergen consisted of homogenized cockroach abdomen extracted with 5 ml of phosphate buffered saline-Tween 20 (PBS-T) extraction buffer. For each allergen, 20 microliters of allergen was placed on glass wool and allowed to coat the fibers. After application of the allergen, 60 microliters of test product was added to the glass wool and allowed to coat the fibers. Finally, 80 microliters of PBS-T was added to extract the allergen. Comparative Composition A was prepared by adding 60 microliters of sterile deionized water instead of test product prior to extraction.

Composition 1 consisted of 2% by weight of a quaternary amine salt mixture in water. The quaternary amine salt mixture consisted of:
32% n-alkyl (50% C14, 40% C12, 10% C16) dimethyl benzyl ammonium chloride;
24% n-octyl decyl dimethyl ammonium chloride;
12% di-n-octyl dimethyl ammonium chloride;
12% di-n-decyl dimethyl ammonium chloride; and
20% inert ingredients

Composition 2 consisted of 2% by weight dimethyl ditallow ammonium chloride in water. The ELISA method for quantification of allergens in micrograms of allergen per ml of sample was then conducted for each extract to determine whether the test products resulted in a deactivation of allergenic proteins. The results are shown in Table 1 below.

TABLE 1

| | Dust mite | Cockroach |
|---|---|---|
| | DER p 1 | Bla g1 |
| | mcg/ml | U/ml |
| Comparative Composition A | 2.86 | 4.00 |
| Composition 1 | 2.96 | 1.13 |
| Composition 2 | 1.41 | <1.12 |

Both Compositions 1 and 2 provided deactivation of cockroach allergenic protein, and Composition 2 provided a deactivation of the dust mite allergenic protein.

### EXAMPLE 2

The allergens used in this experiment were obtained from dog and cat sources. House dust samples from a household with two cats and two dogs was the substrate used for dog and cat allergen. In each case, the dust was treated with either water or allergenic protein deactivating compound in water, allowed to dry and extracted with 5 ml of phosphate buffered saline-Tween 20 (PBS-T) extraction buffer. Comparative Composition B was pure water. Composition 3 consisted of 5% by weight dimethyl ditallow ammonium chloride in water. Composition 4 consisted of 5% by weight didecyl dimethyl ammonium chloride in water. Composition 5 consisted of 5% by weight polymeric quaternium prepared by the polymerization of dimethylamine and bis-dichlorobutene, end capped with tris-hydroxyethylamine in water. Comparative Composition C consisted of 5% by weight active hexadecyl trimethyl ammonium chloride in water.

The ELISA method for quantification of allergens in micrograms of allergen per milliliter or gram of sample (depending upon source) was conducted for each extract to determine whether the test products resulted in a deactivation of allergenic proteins. The results are shown in Table 2 below.

TABLE 2

| | Dog | Cat |
|---|---|---|
| | Can f 1 | Microgram/g |
| | Microgram/ml | Dust |
| Comparative Composition B | 60.4 | 35 |
| Composition 3 | 3.9 | 7 |
| Composition 4 | 19.7 | Nm** |
| Composition 5 | 23.1 | Nm** |
| Comparative Composition C | 63.35 | Nm** |

**“Nm” indicates that the indicated value was not measured.

It is evident from the results in Table 2, that the compounds of this invention provide deactivation of dog and cat allergenic protein.

### EXAMPLE 3

The allergens used in this experiment were obtained from dust mite, dog dander and birch pollen sources, as indicated in Table 3 below. The ELISA method for quantification of allergens from environmental samples was conducted for each of the selected allergens. Whatman GF/C fiberglass filters were used as the matrix for the reactions.

The amount of purified allergen standard applied to each test filter was 25 nanograms (ng)/filter for dust mite, 100 ng/filter for dog, and 50 ng/filter for birch pollen. Filters were prepared by applying these amounts of allergen and allowing the filters to dry at ambient conditions for about 45 minutes. Test filters were produced in duplicate so two sets of testing could be done.

The allergen treated filters were then further treated by adding about 25 mg of test compound via pipette. One control filter (negative control) was prepared without allergen, and test compound was applied thereto. A second control filter (positive control) was prepared with allergen, but was further treated by adding about 25 mg of distilled water instead of test compound.

Each filter was then extracted with phosphate buffered saline, pH 7.4, with 0.05% Tween 20 for 2 hours using the dust extraction protocol and was then tested for levels of allergens.

The results are reported in Table 3 below. Comparative Composition D in Table 3 is the positive control sample (allergen treated with distilled water and not with test compound). Comparative Composition E is the negative control sample (treated with test compound but not with allergen). Composition 6 was the test compound used in this experiment. Composition 6 consisted of 5% by weight methyl bis(modified tallowamido ethyl)-2-hydroxyethyl ammonium methyl sulfate, sold commercially by Stepan Company under the trade name Accosoft® 460HC. The weight of product applied to each filter is also shown in Table 3.

TABLE 3

| | Dust mite | | Dog | | Birch | |
|---|---|---|---|---|---|---|
| | Treatment | | Treatment | | Treatment | |
| | Agent | | Agent | | Agent | |
| | Weight | Dust mite | Weight | Dog | Weight | Birch |
| | Applied | Derf 1 | Applied | Can f 1 | Applied | Bet v1 |
| Sample # | (mg) | ng/Filter | (mg) | ng/Filter | (mg) | ng/Filter |
| Comparative | 24.5 | 26.61 | 25.4 | 67.60 | 25.7 | 53.21 |
| Composition D | | | | | | |
| Composition 6 | 27.8 | 23.86 | 24.8 | 74.50 | 25.5 | 25.11 |
| (Run 1) | | | | | | |
| Composition 6 | 29.0 | 21.30 | 24.5 | 67.91 | 24.6 | 27.70 |
| (Run 2) | | | | | | |
| Comparative | 26.7 | ND | 25.9 | ND*** | 26.8 | ND*** |
| Composition E | | | | | | |

***“ND” indicates that the indicated value was below the detection capability limits.

As can be determined with respect to the results reported above in Table 3, the average amount of dust mite allergen for the two runs of Composition 6 was 22.6 ng/filter after treatment, which is a 15% reduction in amount as compared to the results for Comparative Compound D (the positive control sample). Additionally, the average amount of birch pollen allergen for the two runs of Composition 6 was 26.4 ng/filter, which is a 50.4% reduction in amount as compared to the results for Comparative Compound D.

### EXAMPLE 4

This experiment was designed to test the anti-allergenic capabilities of one composition comprising an allergenic deactivation compound, as well as at least one surface treating antimicrobial agent and at least one treating antimicrobial agent.

The allergens used in this experiment were obtained from dust mite, dog dander and birch pollen sources, as indicated in Table 4 below. The ELISA method for quantification of allergens from environmental samples was conducted for each of the selected allergens. Whatman GF/C fiberglass filters were used as the matrix for the reactions.

The amount of purified allergen standard applied to each test filter was 25 nanograms (ng)/filter for dust mite, 100 ng/filter for dog, and 50 ng/filter for birch pollen. Filters were prepared by applying these amounts of allergen and allowing the filters to dry at ambient conditions for about 45 minutes. Test filters were produced in duplicate so two sets of testing could be done.

The allergen treated filters were then further treated by adding about 25 mg of test compound via pipette. One control filter (negative control) was prepared without allergen, and test compound was applied thereto. A second control filter (positive control) was prepared with allergen, but was further treated by adding about 25 mg of distilled water instead of test compound.

Each filter was then extracted with phosphate buffered saline, pH 7.4, with 0.05% Tween 20 for 2 hours using the dust extraction protocol and was then tested for levels of allergens.

The results are reported in Table 4 below. The weight of product applied to each filter is also shown in Table 4. Comparative Composition F in Table 4 is the positive control sample (allergen treated with distilled water and not with test compound). Comparative Composition G is the negative control sample (treated with test compound but not with allergen). Composition 7 was the test compound used in this experiment. Composition 7 consisted of:
5.5% by weight methyl bis(modified tallowamido ethyl)-2-hydroxyethyl ammonium methyl sulfate, sold commercially by Stepan Company under the trade name Accosoft® 460HC,
0.4% by weight of 50% n-alkyl dimethyl benzyl ammonium chlorides and n-alkyl dimethyl ethylbenzyl ammonium chlorides, sold commercially by Stepan Company under the trade name BTC 2125® M,
53% by weight isoproponal,
6% by weight triethylene glycol, and

35.1% by weight water.

TABLE 4

| | Dust mite | | Dog | | Birch | |
|---|---|---|---|---|---|---|
| | Treatment | | Treatment | | Treatment | |
| | Agent | | Agent | | Agent | |
| | Weight | Dust mite | Weight | Dog | Weight | Birch |
| | Applied | Derf 1 | Applied | Can f 1 | Applied | Bet v1 |
| Sample # | (mg) | ng/Filter | (mg) | ng/Filter | (mg) | ng/Filter |
| Comparative | 25.1 | 23.63 | 24.0 | 72.05 | 24.7 | 46.25 |
| Composition F | | | | | | |
| Composition 7 | 20.6 | 20.84 | 22.6 | 90.59 | 22.3 | 35.30 |
| (Run 1) | | | | | | |
| Composition 7 | 20.2 | 17.35 | 20.7 | 63.11 | 21.8 | 31.74 |
| (Run 2) | | | | | | |
| Comparative | 20.7 | ND*** | 19.7 | ND*** | 21.0 | ND*** |
| Composition G | | | | | | |

***“ND” indicates that the indicated value was below the detection capability limits.

As can be determined with respect to the results reported above in Table 4, the average amount of dust mite allergen for the two runs of Composition 7 was 19.1 ng/filter after treatment, which is a 19.2% reduction in amount as compared to the results for Comparative Compound F (the positive control sample). Additionally, the average amount of birch pollen allergen for the two runs of Composition 7 was 33.52 ng/filter after treatment, which is a 27.5% reduction in amount as compared to the results for Comparative Compound F.

The presently described technology and the manner and process of making and using it, are now described in such full, clear, concise and exact terms as to enable one of ordinary skill in the art to which the present technology pertains, to make and use the same. It should be understood that the foregoing describes some embodiments and advantages of the invention and that modifications may be made therein without departing from the spirit and scope of the presently described technology as set forth in the claims. Moreover, the present technology has been described with reference to preferred and alternate embodiments. Modifications and alterations will occur to others upon the reading and understanding of the specification. It is intended to include all such modifications and alterations insofar as they come within the scope of the appended claims or equivalents thereof. To particularly point out and distinctly claims the subject matter regarded as the invention, the following claims conclude this specification.

What is claimed is:

1. A method for deactivating the allergenic proteins on a surface comprising the step of exposing the surface to an allergenic protein deactivating formulation comprising an allergenic protein deactivating compound comprising poly-functional nitrogen containing compounds, mono-functional nitrogen-containing compounds, poly-functional cation-containing compounds, mono-functional cation-containing compounds, combinations thereof, or derivatives thereof.

2. The method of claim 1, wherein the allergenic protein deactivating compound is selected from the group consisting essentially of dimethyl dialkyl ammonium salts, methyl trialkyl ammonium salts, tetraalkyl ammonium salts, tetramethyl ammonium salts, imidazoline based quaternaries, benzyl alkyl dimethyl ammonium salts, benzyl methyl dialkyl ammonium salts, benzyl trialkyl ammonium salts, dibenzyl dimethyl ammonium salts, propoxylated quaternary amines, ethoxylated quaternary amines, dialkyl diethoxylated ammonium salts, dipropoxylated ammonium salts, alkyl methyl diethoxylated ammonium salts, dipropoxylated ammonium salts, tetraethoxylated ammonium salts, tetrapropoxylated ammonium salts, methyl triethoxylated ammonium salts, propoxylated ammonium salts, ester quaternaries, phosphate-containing quaternary salts, pyridinium salts, polyvinyl pyridinium salts, vinyl pyridinium copolymer salts, nitrogen-containing heterocyclic amine salts, non-cellulose-based polyquaternium salts, vinyl pyrrolidone copolymers, and combinations thereof.

3. The method of claim 1, wherein the allergenic protein deactivating formulation further comprises an additive selected from the group consisting of surfactants, buffers, oils, waxes, solvents, preservatives, cyclodextrins, soluble aluminum salts, urea, alkyl trimethyl ammonium salts, polyvinyl pyrrolidone, tannic acid, immobilized tannic acid, polyphenols, enzymes, benzyl benzoate, inorganic salts and mixtures thereof.

4. The method of claim 1, wherein the allergenic protein deactivating compound is selected from the group consisting essentially of dimethyl dialkyl ammonium salts, benzyl trialkyl ammonium salts, methyl trialkyl ammonium salts, tetraalkyl ammonium salts, tetramethyl ammonium salts, imidazoline-based quaternaries, benzyl alkyl dimethyl ammonium salts, benzyl methyl dialkyl ammonium salts, benzyl trialkyl ammonium salts, dibenzyl dimethyl ammonium salts, propoxylated quaternary amines, ethoxylated quaternary amines, dialkyl diethoxylated ammonium salts, dipropoxylated ammonium salts; methyl alkyl diethoxylated ammonium salts, methyl alkyl dipropoxylated ammonium salts, or benzyl alkyl diethoxylated ammonium salts, benzyl alkyl dipropoxylated ammonium salts, benzyl triethoxylated ammonium salts, benzyl tripropoxylated ammonium salts, methyl triethoxylated ammonium salts, methyl tripropoxylated ammonium salts, ester quaternaries, phosphate-containing quaternaries, pyridinium salts, polyvinyl pyridinium salts, vinyl pyridinium copolymer salts, nitrogen-containing heterocyclic amine salts, non-cellulose-based polyquaternium salts, derivatives thereof, and mixtures thereof.

5. A product produced according to method of claim 1.

6. The method of claim 1, wherein the allergenic protein deactivating compound is a quaternary ammonium salt.

7. The method of claim 6, wherein the quaternary ammonium salt is dimethyl dialkyl ammonium chloride.

8. The method of claim 6, wherein the allergenic protein deactivating formulation further comprises at least one antimicrobial agent.

9. An allergenic protein deactivating formulation comprising an allergenic protein deactivating compound selected from the group consisting essentially of sulfobetaines, betaines, amino carboxylates, amine oxides, amides, nitrogen-containing heterocyclic amides, dimethyl dialkyl ammonium salts, methyl trialkyl ammonium salts, tetraalkyl ammonium salts, tetramethyl ammonium salts, imidazoline-based quaternaries, benzyl alkyl dimethyl ammonium salts, benzyl methyl dialkyl ammonium salts, benzyl trialkyl ammonium salts, dibenzyl dimethyl ammonium salts, propoxylated quaternary amines, ethoxylated quaternary amines, dialkyl diethoxylated ammonium salts, dipropoxylated ammonium salts, alkyl methyl diethoxylated ammonium salts, dipropoxylated ammonium salts, tetraethoxylated ammonium salts, tetrapropoxylated ammonium salts, methyl triethoxylated ammonium salts, tripropoxylated ammonium salts, ester quaternaries, phosphate-containing quaternaries, pyridinium salts, polyvinyl pyridinium salts, vinyl pyridinium copolymer salts, nitrogen-containing heterocyclic amine salts, non-cellulose-based polyquaternium salts, amino carboxylates, amine oxides, amides, nitrogen-containing heterocyclic amides, vinyl pyrrolidone copolymers, derivatives thereof, and combinations thereof

10. The allergenic protein deactivating formulation of claim 9, comprising about 5% or less by weight of the allergenic protein deactivating compound in a solvent, a diluent, or an aqueous delivery composition.

11. The allergenic protein deactivating formulation of claim 9, comprising about 2.5% or less by weight of the allergenic protein deactivating compound in a solvent, a diluent, or an aqueous delivery composition.

12. The allergenic protein deactivating formulation of claim 9, comprising about 1% or less by weight of the allergenic protein deactivating compound in a solvent, a diluent, or an aqueous delivery composition.

13. The allergenic protein deactivating formulation of claim 9, comprising about 0.5% or less by weight of the allergenic protein deactivating compound in a solvent, a diluent, or an aqueous delivery composition.

14. The allergenic protein deactivating formulation of claim 9, comprising about 0.2% or less by weight of the allergenic protein deactivating compound in a solvent, a diluent, or an aqueous delivery composition.

15. The allergenic protein deactivating formulation of claim 9, comprising about 0.05% or less by weight of the allergenic protein deactivating compound in a solvent, a diluent, or an aqueous delivery composition.

16. The allergenic protein deactivating formulation of claim 9, wherein the solvent, diluent, or aqueous delivery composition is a member selected from the group consisting of water, alcohols, glycols, and organic solvents.

17. The method of claim 16, wherein the solvent, diluent, or aqueous delivery composition is water.

18. The allergenic protein deactivating formulation of claim 9, further comprising an additive selected from the group consisting of cyclodextrins, soluble aluminum salts, urea, alkyl trimethyl ammonium salts, polyvinyl pyrrolidone, tannic acid, immobilized tannic acid, polyphenols, enzymes, benzyl benzoate, inorganic salts, surfactants, buffers, oils, waxes, solvents, preservatives and mixtures thereof.

19. The allergenic protein deactivating formulation of claim 9, wherein the allergenic protein deactivating compound comprises at least about 0.05% by weight of a quaternary amine salt mixture comprising:
at least about 32% by weight n-alkyl (50% C14, 40% C12, 10% C16) dimethyl benzyl ammonium chloride;
at least about 24% by weight n-octyl decyl dimethyl ammonium chloride;
at least about 12% by weight di-n-octyl dimethyl ammonium chloride; and
at least about 12% by weight di-n-decyl dimethyl ammonium chloride.

20. The allergenic protein deactivating formulation of claim 9, wherein the allergenic protein deactivating compound comprises at least about 0.05% by weight dimethyl ditallow ammonium chloride.

21. The allergenic protein deactivating formulation of claim 9, wherein the allergenic protein deactivating compound comprises at least about 0.05% by weight of a non-cellulose-based polymeric quaternary amine salt.

22. The allergenic protein deactivating formulation of claim 9, wherein the allergenic protein deactivating compound comprises at least about 0.05% by weight dimethyl didecyl ammonium chloride.

23. The allergenic protein deactivating formulation of claim 9, wherein the allergenic protein deactivating compound comprises at least about 0.2% by weight of a quaternary amine salt mixture comprising:
at least about 32% by weight n-alkyl (50% C14, 40% C12, 10% C16) dimethyl benzyl ammonium chloride;
at least about 24% by weight n-octyl decyl dimethyl ammonium chloride;
at least about 12% by weight di-n-octyl dimethyl ammonium chloride; and
at least about 12% by weight di-n-decyl dimethyl ammonium chloride.

24. The allergenic protein deactivating formulation of claim 9, wherein the allergenic protein deactivating compound comprises at least about 0.2% by weight dimethyl ditallow ammonium chloride.

25. The allergenic protein deactivating formulation of claim 9, wherein the allergenic protein deactivating compound comprises at least about 0.2% by weight of a non-cellulose-based polymeric quaternary amine salt.

26. The allergenic protein deactivating formulation of claim 9, wherein the allergenic protein deactivating compound comprises at least about 0.2% by weight dimethyl didecyl ammonium chloride.

27. The allergenic protein deactivating formulation of claim 9, wherein the allergenic protein deactivating compound is selected from the group consisting essentially of dimethyl dialkyl ammonium salts, benzyl trialkyl ammonium salts, methyl trialkyl ammonium salts, tetraalkyl ammonium salts, tetramethyl ammonium salts, imidazoline-based quaternaries, benzyl alkyl dimethyl ammonium salts, benzyl methyl dialkyl ammonium salts, benzyl trialkyl ammonium salts, dibenzyl dimethyl ammonium salts, propoxylated quaternary amines, ethoxylated quaternary amines, dialkyl diethoxylated ammonium salts, dipropoxylated ammonium salts; methyl alkyl diethoxylated ammonium salts, methyl alkyl dipropoxylated ammonium salts, or benzyl alkyl diethoxylated ammonium salts, benzyl alkyl dipropoxylated ammonium salts, benzyl triethoxylated ammonium salts, benzyl tripropoxylated ammonium salts, methyl triethoxylated ammonium salts, methyl tripropoxylated ammonium salts, ester quaternaries, phosphate-containing quaternaries, pyridinium salts, polyvinyl pyridinium salts, vinyl pyridinium copolymer salts, nitrogen-containing heterocyclic amine salts, non-cellulose-based polyquaternium salts, derivatives thereof, and mixtures thereof.

28. The allergenic protein deactivating formulation according to claim 9, wherein allergenic protein deactivation composition is a quaternary ammonium salt.

29. The allergenic protein deactivation formulation according to claim 28, wherein the quaternary ammonium salt is dimethyl dialkyl ammonium chloride.

30. The allergenic protein deactivation formulation of claim 9, further comprising at least one antimicrobial agent.

31. The allergenic protein deactivation formulation of claim 30, wherein the at least one antimicrobial agent is a surface treating antimicrobial agent.

32. The allergenic protein deactivation formulation of claim 31, wherein the surface treating antimicrobial agent is selected from the group consisting of quaternary ammonium compounds, sodium alkylbenzene sulfonates, mercury compounds, halogens, halogen compounds, aldehydes, phenols, isothiazonlines, alcohols, carbamates, halide compounds, peroxides, parabens, iodine, metals, peracids, carbonates, hypochlorites, chloramines, benzalkonium chloride, derivatives thereof, and mixtures thereof.

33. The allergenic protein deactivation formulation of claim 30, wherein at least one antimicrobial agent is an air treating antimicrobial agent.

34. The allergenic protein deactivation formulation of claim 33, wherein the air treating antimicrobial agent is selected from the group consisting of propylene glycol, dipropylene glycol, triethylene glycol, derivatives thereof, and mixtures thereof.

35. A method for deactivating the allergenic proteins on a surface comprising the step of exposing the surface to an allergenic protein deactivating formulation further comprising an allergenic protein deactivating compound, wherein the allergenic protein deactivating compound comprises a concentration of about 5% by weight or less of a particulate material in a solvent, a diluent, or an aqueous delivery composition.

36. The method of claim 35, wherein the particulate material is a member selected from the group consisting of poly-functional nitrogen containing compounds, mono-functional nitrogen-containing compounds, poly-functional cation-containing compounds, mono-functional cation-containing compounds, combinations thereof, and derivatives thereof.

37. The method of claim 35, wherein the allergenic protein deactivating compound comprises a concentration of about 2.5% or less by weight of the particulate material in the solvent, the diluent, or the aqueous delivery composition.

38. The method of claim 35, wherein allergenic protein deactivating compound comprises a concentration of about 1.5% or less by weight of the particulate material in the solvent, the diluent, or the aqueous delivery composition.

39. The method of claim 35, wherein the solvent, the diluent, or the aqueous delivery composition is a member selected from the group consisting of water, alcohols, glycols, and organic solvents.

40. The method of claim 39, wherein the solvent, the diluent, or the aqueous delivery composition is water.
